(19) United States
(12) Patent Application Publication
(10) Pub. No.: US 2014/0348959 A1
(43) Pub. Date: Nov. 27, 2014

(54) Formulations and Methods for Treating Ear Conditions

(71) Applicant: Particle Sciences, Inc., Bethlehem, PA (US)

(72) Inventors: Mark A. Mitchnick, East Hampton, NY (US); Garry Gwozdz, Bethlehem, PA (US)

(21) Appl. No.: 14/457,637

(22) Filed: Aug. 12, 2014

Related U.S. Application Data
(63) Continuation of application No. PCT/US13/26213, filed on Feb. 14, 2013.
(60) Provisional application No. 61/598,405, filed on Feb. 14, 2012.

(51) Int. Cl. A61K 36/61; A61K 31/47; A61K 31/4152; A61K 31/245; A61K 45/06; A61K 31/122; A61K 31/085; A61K 31/045; A61K 36/235; A61K 31/5375; A61K 31/167

(52) U.S. Cl. CPC A61K 36/61; A61K 31/5375; A61K 31/47; A61K 31/4152; A61K 31/245; A61K 31/167; A61K 31/122; A61K 31/085; A61K 31/045; A61K 36/235; A61K 45/06
USPC 424/725; 514/239.2; 514/312; 514/404; 514/536; 514/626; 514/691; 514/720; 514/739

(57) ABSTRACT

Compositions and methods for treatment of conditions such as ear pain are disclosed.

### RELATED APPLICATION

This application is a Continuation Application of International Application No. PCT/US13/26213 filed Feb. 14, 2013, which claims the benefit of and priority to U.S. Provisional Patent Application No. 61/598,405, filed Feb. 14, 2012, the contents of which are incorporated herein by reference in their entirety.

### BACKGROUND OF THE INVENTION

Otitis media, an infection of the middle ear, affects millions of children and adults each year. In addition to causing pain and fever, ear infections can also result in permanent hearing loss.

Bacterial otitis media is typically treated with antibiotics, which are frequently administered orally. In addition, palliative treatment only, via the use of topical anesthetics and oral pain medication such as acetaminophen, may be used.

Although palliative formulations for administration to the ear canal have been reported, such formulations, which are often formulated with an oily base material, may be messy or inconvenient to apply. In addition, leakage of the formulation from the ear canal after application may reduce the efficacy of the treatment by reducing the effective amount applied, and, importantly, by reducing compliance as the mess is a deterrent for parents and children.

Improved formulations and methods for treating otitis media and other conditions of the ear are needed.

### SUMMARY OF THE INVENTION

The present invention relates generally to compositions and methods for treatment of conditions such as ear pain.

In one aspect, the invention relates to a topical formulation comprising:

(a) an effective pain-reducing amount of an anesthetic or analgesic; and

(b) a pharmaceutically acceptable carrier;

wherein the topical formulation is substantially non-flowable after application to an ear canal of a subject.

In certain embodiments, the anesthetic or analgesic is selected from the group consisting of antipyrine, benzocaine, lidocaine, butamben, dibucaine, oxybuprocaine, pramoxine, proparacaine, proxymetacaine, and tetracaine. In certain embodiments, the anesthetic is an essential oil having anesthetic properties, or an active component of such an essential oil. In certain embodiments, the essential oil is clove oil or fennel oil. In certain embodiments, the active component is eugenol, linalool, or fenchone, or a mixture thereof.

In certain embodiments, the composition further comprises a second therapeutic agent; in certain embodiments, the second therapeutic agent is selected from the group consisting of: antibiotics, antivirals, antifungals, antiseptics, astringents, anti-irritants, anti-inflammatories, and agents to aid in the softening or removal of cerumen.

In certain embodiments, the active ingredient or ingredients are present as a particle, capsule or solution, or a combination thereof.

In certain embodiments, the formulation has a viscosity of at least about 10,000 cps.

In certain embodiments, the anesthetic is dispersed or dissolved in a volatile carrier or base. In certain embodiments, the volatile carrier comprises a material selected from the group consisting of volatile silicones, alcohols, biodegradable polymers, or volatile oils. In certain embodiments, the volatile base comprises cyclomethicone 5.

In certain embodiments, the formulation is shear thinning such that the formulation can be thinned for application by shaking and thickens when applied to the ear canal.

In certain embodiments, the formulation thickens upon application to the ear canal.

In certain embodiments, the formulation has a low (or lower) viscosity when chilled and is substantially non-flowable (i.e., has a higher viscosity) at body temperature.

In certain embodiments, the formulation comprises a viscosity adjusting agent, wherein the viscosity adjusting agent comprises a polymer which exhibits a thermal gelation response. In certain embodiments, the viscosity adjusting agent comprises a polymer selected from the group consisting of chitosan, cellulosic derivatives, gelatin, N-isopropylacrylamide polymers, poly(ethylene oxide)-b-poly(propylene oxide)-b-poly(ethylene oxide) polymers and poly(ethylene glycol)-biodegradable polyester copolymers.

In certain embodiments, the formulation further comprises a drying agent.

In another aspect, the invention provides a topical formulation comprising an anesthetic, wherein the formulation provides anesthesia for at least about 4 hours after application to an ear canal of a subject.

In another aspect, the invention provides a topical formulation, the formulation comprising an anesthetic, wherein the formulation has an onset of action of less than about 10 minutes after application to an ear canal of a subject.

In certain embodiments of any of the formulations of the invention, the formulation is essentially free of zinc.

In certain embodiments of any of the formulations of the invention, the formulation is essentially free of additional penetration enhancers.

In certain embodiments of any of the formulations of the invention, the formulation is essentially free of additional antibiotics.

In certain embodiments of any of the formulations of the invention, the formulation is essentially free of additional preservatives.

In another aspect, the invention provides a method of treating ear pain or other conditions of the external, middle or inner ear, or other bodily organs or structures accessible from the ear canal, the method comprising administering a composition of the invention to an ear canal of a subject in need of such treatment.

In certain embodiments of the methods of the invention, after 5 minutes after application, the composition does not flow from the ear canal for at least about 10 minutes.

In certain embodiments, the ear pain is pain due to otitis.

In another aspect, the invention provides a method of treating ear pain or other conditions of the external, middle or inner ear, or other bodily organs or structures accessible from the ear canal, the method comprising administering a composition of the invention to an ear canal of a subject in need of such treatment, under conditions such that leakage of the composition from the ear canal is minimized without plugging the ear canal.

In another aspect, the invention provides a topical formulation consisting essentially of:

(a) an effective pain-reducing amount of an anesthetic or analgesic; and

(b) a pharmaceutically acceptable carrier;

wherein the topical formulation is substantially non-flowable after application to an ear canal of a subject.

In another aspect, the invention provides a topical formulation comprising:

(a) an effective pain-reducing amount of an anesthetic or analgesic; and

(b) a pharmaceutically acceptable carrier;

wherein the topical formulation does not leak from the ear canal of a subject after application of the formulation to the ear canal of the subject.

Other details, aspects and embodiments of the invention will be apparent from the description herein.

### DETAILED DESCRIPTION OF THE INVENTION

The present invention relates to formulations of topical anesthetics that are formulated in such a way as to prevent or minimize leakage from the ear canal after administration. Methods of using such formulations are also disclosed. The formulations and methods of the invention can increase patient compliance and reduce the substantial suffering associated with middle ear infections.

Thus, in one aspect, the invention relates to a topical formulation that is substantially non-flowable after application to an ear canal of a subject.

In one aspect, the invention relates to a topical formulation comprising:

(a) an effective pain-reducing amount of an anesthetic or analgesic; and

(b) a pharmaceutically acceptable carrier;

wherein the topical formulation is substantially non-flowable after application to an ear canal of a subject.

Topical anesthetics and analgesics known in the art can be used in the formulations of the invention. Examples of topical anesthetics and analgesics include antipyrine, benzocaine, lidocaine, butamben, dibucaine, oxybuprocaine, pramoxine, proparacaine, proxymetacaine, tetracaine, chloroprocaine, cocaine, cyclomethycaine, dimethocaine/larocaine, piperocaine, propoxycaine, procaine/novocaine, articaine, bupivacaine, etidocaine, levobupivacaine, mepivacaine, prilocaine, ropivacaine, methohexital, ketamine, prilocaine, thiopental, propofol, and trimecaine; essential oils, such as clove oil or fennel oil, or active components of such essential oils, such as menthol, eugenol, linalool, or fenchone; or mixtures of these topical anesthetics and analgesics. Exemplary anesthetics include articaine-epinephrine and antipyrine-benzocaine

Topical formulations according to the invention are substantially non-flowable after application to an ear canal of a subject. The term “substantially non-flowable”, as used herein, refers to a composition that does not significantly flow or leak from the ear canal, after an initial period of administration (e.g., of about 10 minutes or less), for at least about 10 minutes (or at least about 30 minutes, one hour, and the like). Thus, after a formulation of the invention is instilled into the ear canal, the formulation is, or becomes, thickened, viscous, or otherwise non-flowable, and remains in the ear canal, substantially without leakage (e.g., less than 10% leakage of the anesthetic or analgesic from the ear canal), for at least about 10 minutes (or at least about 30 minutes, one hour, and the like).

A formulation can be tested to determine whether the formulation is “substantially non-flowable” according to a variety of methods, some of which are known in the art. For example, the viscosity of a formulation can be tested by known methods, e.g., by any method, such as use of a standard oscillatory rheometer. A formulation having a viscosity of at least about 10,000 centipoise (cps) at body temperature (i.e., about 35-40° C.) will generally be “substantially non-flowable” as used herein. Another test is to place the formulation in a simulated ear canal, and position the opening of the simulated ear canal downward, measuring the amount of formulation that leaks from the simulated ear canal over a measured period of time. A substantially non-flowable composition will not leak from the opening of the simulated ear canal for at least about 10 minutes (or at least about 30 minutes, one hour, and the like).

A substantially non-flowable composition can be provided in a number of ways. For example, a composition can be made more viscous by the addition of conventional viscosity modifiers (also referred to herein as “viscogenic agents”), as described herein. Alternatively, a composition can be made viscous under certain conditions (e.g., conditions generally prevailing in the ear canal) and flowable under different conditions (e.g., higher or lower temperature, shear conditions, etc.), so that a composition can be administered to the ear canal in a flowable condition and become substantially non-flowable in the ear canal. Substantially non-flowable compositions can also be prepared by using a volatile solvent as a carrier; when the composition is instilled into the ear canal, body temperature causes the volatile carrier to evaporate, leaving a thick, substantially non-flowable residue in the ear canal. In further embodiments, compositions can be rendered substantially non-flowable in situ by the addition of a material that causes the applied composition to gel or thicken after application to the ear canal.

### Formulations Having High Viscosity

In certain embodiments, a formulation of the invention can be prepared with an analgesic and/or anesthetic dissolved or suspended in a carrier which can include a viscosity modifier or modifiers, so as to provide a formulation having a viscosity of at least 1,000, 10,000, 20,000, 30,000, 40,000, 50,000, 60,000 centipoise or more. As used herein, the terms “viscosity modifier” or “viscogenic agent” refer to a polymer or other chemical moiety that increases the viscosity of a fluid. Examples of viscosity modifiers include chitosan, cellulosic derivatives such as methyl cellulose, gelatin, N-isopropylacrylamide polymers, poly(ethylene oxide)-b-poly(propylene oxide)-b-poly(ethylene oxide) polymers and poly(ethylene glycol)-biodegradable polyester copolymers, alginic acid, hyaluronic acid, acacia (gum Arabic) carbomer, cetostearyl alcohol, particulates such as silica, and the like, or a combination of viscosity modifiers.

Exemplary viscogenic agents include gellan (e.g., sold under the trade name GELRITE or KELCOGEN), CARBOPOL 940 (polyacrylic acid) with hydroxypropylmethylcellulose (HPMC), N-isopropyl acrylamide (NiPAAm) with sodium acrylate and n-N-alkylacrylamide, polyacrylic acid with polyethylene glycol (PEG) or polymethacrylic acid with PEG, cellulose acetate hydrogen phthalate latex (CAP), sodium alginate, and nonionic surfactants such as poloxamers (e.g., sold under the trade name PLURONIC) and polyoxamine (e.g., sold under the trade name TETRONIC) reversible temperature-dependent gelling systems. Gellan is a natural polymer, anionic deacetylated exocellular polysaccharide, secreted by Pseudomonas elodea. The tetrasaccharide repeating unit consists of one alpha-L-rhamnose, one beta-D-glucuronic acid, and two beta-D-glucose moieties. The in situ gelling mechanism of gellan is cation-induced (e.g., presence of calcium ions) and temperature-dependent (e.g., physiologic temperature). Gelation is thermally reversible. CARBOPOL 940 with HPMC gels in situ in a pH-dependent manner. CARBOPOL is the gelling agent and the HPMC is used to enhance the viscosity of the gel. NiPAAm with sodium acrylate and n-N-alkylacrylamide is a terpolymer hydrogel that can undergo a temperature based reversible sol-gel transformation. Sodium acrylate and n-N-alkylacrylamide are used to modify the properties of the hydrogel, and in particular, the transition temperature. Polyacrylic acid can be dissolved in hydroalcoholic solution and after being injected, the alcohol diffuses out causing the polymers to precipitate and gelling of the solution. CAP is a nanoparticulate system that gels in a pH-dependent manner. Sodium alginate gels in the presence of calcium or other polyvalent ions.

Nonionic surfactants such as poloxamers and poloxamines can also be used. Poloxamers are well known in the pharmaceutical arts and are described, for example, by Irving R. Schmolka, Poloxamers in the Pharmaceutical Industry, in Polymers for Controlled Drug Delivery, Chapter 10 (Peter J. Tarcha ed., 1990). Poloxamers are triblock copolymers because they are composed of two different polymer blocks (i.e., hydrophilic poly(oxyethylene) blocks and hydrophobic poly(oxypropylene) blocks) configured as a triblock of poly(oxyethylene)-poly(oxypropylene)-poly(oxyethylene). Poloxamers are one class of block copolymer surfactants having a propylene oxide block hydrophobe and an ethylene oxide hydrophile. Poloxamers are commercially available (e.g., PLURONIC polyols are available from BASF Corporation). Alternatively, polaxamers can be synthesized by known techniques.

Aqueous formulations of poloxamers exhibit reverse thermal gelation, or reverse thermosetting. When an aqueous poloxamer formulation is heated over its gelation temperature, its viscosity increases and it transforms into a gel. When an aqueous poloxamer formulation is cooled below its gelation temperature, its viscosity decreases and it transforms into a liquid. The transition between gel and liquid does not involve a change in the chemical composition of the formulation, and is reversible and repeatable. The gel-liquid transition temperature of an aqueous poloxamer formulation can be adjusted by one of ordinary skill in the art using routine experimentation (e.g., by manipulating poloxamer concentration, pH and presence of other ingredients in the formulation). In some embodiments, compositions have a gelation temperature that is greater than the ambient temperature and less than or equal to the temperature of the tympanic membrane. Such compositions can be conveniently applied via an individual's ear canal as a liquid and then can transform into a gel against the tympanic membrane, thereby maintaining an active agent in the formulation in close proximity to the tympanic membrane.

The amount of viscosity modifier used will depend on the viscosity modifier and the other components of the formulation, and on the desired viscosity of the formulation; exemplary amounts of viscosity modifier range from 0.1%-25% w/w. In certain embodiments, a viscosity modifer (or combination of viscosity modifiers), when included in a composition of the invention, allow the composition to transform from a liquid-like state (e.g., flowable) at 25° C. to a solid-like state (e.g., a gel) after contact with the tympanic membrane, and can be non-biodegradable, i.e., not broken down by chemicals or enzymes naturally present in a mammal, or biodegradable. Compositions include an amount of viscogenic agent effective to yield a viscosity of the composition of less than 100,000 cps at 25° C. (e.g., less than 90,000, 60,000, 30,000, 20,000, or 10,000 cps) and, generally, a minimum yield stress of 39 Pa after application to the tympanic membrane. Typically, a composition includes 0.05 to 50% of a viscogenic agent (e.g., 0.15 to 25, 5 to 45, 10 to 40, 12 to 37, 15 to 35, 17 to 33, or 20 to 30% of a viscogenic agent).

In certain embodiments, a viscosity modifier can be added after the formulation is applied to or instilled into the ear canal, to cause a flowable composition to become substantially non-flowable in situ. For example, materials are known that can be made to gel in situ by the addition of and reaction with a second material. An example of such a system would be administration to the ear canal of a flowable formulation comprising alginate, followed by thickening or gellation of the alginate formulation by addition of divalent cations such as Ca²⁺ (e.g., as calcium lactate or calcium gluconate, e.g., as a solution). Alternatively, a flowable formulation comprising divalent cations such as Ca²⁺ could be administered to the ear canal, followed by thickening or gellation of the formulation with an alginate solution.

### Shear Thinning Formulations

In certain embodiments, a shear responsive formulation is provided, in which the formulation can be thinned for application by shaking, or by drawing the formulation into an applicator device, and wherein the formulation thickens after application to the ear canal. Thus, the formulation includes, in addition to the analgesic and/or anesthetic and any carrier, a shear responsive material in an amount sufficient to cause thinning of the formulation (e.g., to a viscosity of about 10-1000 cps) when shaken or perturbed, and thickening of the formulation when not under shear conditions (e.g., a viscosity of about 1,000-50,000 cps or 10,000-50,000 cps or more when applied to and at rest in the ear canal).

Examples of shear responsive materials include carbomers such as Carbomer 934P, and hydroxyethylcellulose, or combinations thereof. The amount of shear responsive material used will depend on the shear responsive material and the other components of the formulation, and on the desired viscosity of the formulation (both under shear conditions and at rest); exemplary amounts include 0.05%-0.15% w/w Carbomer 934P, or 2% w/w hydroxyethylcellulose. Another approach to achieve shear responsive behavior is by formulation of a transiently structured particulate suspension using a material such as fumed silica.

### Thermal Gelling Formulations

In certain embodiments, a thermal gelling formulation is provided, in which the formulation can be thinned for application by chilling, and then thickens when warmed, e.g., when applied to the ear canal, or that thins when warmed to a temperature above normal body temperature and thickens upon cooling to body temperature. Thus, the formulation includes, in addition to the analgesic and/or anesthetic and any carrier, a thermal gelling material (a material that exhibits a thermal gelation response) in an amount sufficient to cause thinning of the formulation (e.g., to a viscosity of about 10-1000 cps) when chilled (or warmed) to a first temperature, and thickening of the formulation (e.g., to a viscosity of about 1,000-50,000 cps or 10,000-50,000 cps when applied to and at rest in the ear canal) when warmed (or cooled) to a second temperature, which may be body temperature, or about 37° C. In certain embodiments, the formulation has a low viscosity when chilled and is substantially non-flowable at body temperature.

Examples of thermal gelling materials include naturally-occurring polymers such as chitosan, cellulosic derivatives, and gelatin, or modified or derivatized forms of such polymer, as well as synthetic thermally responsive materials such as N-isopropylacrylamide polymers, poly(ethylene oxide)-b-poly(propylene oxide)-b-poly(ethylene oxide) polymers and poly(ethylene glycol)-biodegradable polyester copolymers, or combinations thereof. The amount of thermal gelling material used will depend on the thermal gelling material and the other components of the formulation, and on the desired viscosity of the formulation (both under low and high temperature conditions); exemplary amounts include 0-40% w/w of a thickener such as Poloxamer 407.

### Formulations Having a Volatile Base

In certain embodiments, a formulation according to the invention includes an anesthetic and/or analgesic compound dispersed or dissolved in a volatile carrier. A “volatile carrier” or “volatile base”, as used herein, refers to a compound or material capable of dispersing, suspending, or dissolving the anesthetic and/or analgesic compound, and having a vapor pressure at 37° C. such that at least about 25% of the volatile carrier will evaporate when the formulation is administered to the ear canal. Upon evaporation of the volatile carrier, the anesthetic and/or analgesic compound will be deposited in the ear as a residue or film in the ear canal. In certain embodiments, the volatile carrier has a boiling point of less than 50° C., or 40° C., or 30° C.

Examples of suitable volatile carriers include comprises a material selected from the group consisting of volatile silicones (such as cyclomethicone 5), alcohols (such as lower alkanols or fluoroalkanols), volatile oils, or other materials having a vapor pressure such that, at approximately body temperature, the volatile material can evaporate, e.g., at a rate such that at least about 25% w/w of the volatile material will evaporate in 10 minutes. Thus, in certain embodiments, at least about 25% of the volatile carrier will evaporate within about 2, 5, or 10 minutes of administration of the composition to the ear canal or tympanic membrane. The amount of volatile carrier used will depend on the volatile carrier material and the other components of the formulation, and on the desired rate of evaporation of the formulation (both under low and high temperature conditions); exemplary amounts include 10-99% w/w volatile silicones, such as cyclomethicone 5.

It will be appreciated that formulations according to the invention can include a combination of, e.g., shear responsive or thermally responsive materials together with volatile carriers, to provide a formulation that minimizes residual material in the ear canal.

### Additional Components of Formulations

Formulations of the invention can optionally include a second (or third, fourth, or more) therapeutic agent for treating conditions of the external, middle or inner ear, or other structures accessible from the ear canal. Examples of such additional therapeutic agents include antibiotics (for example, quinolone antibiotics, penicillin antibiotics (such as amoxicillin), macrolide antibiotics (such as azithromycin), cephalosporin antibiotics, sulfa antibiotics, beta-lactamase inhibitors, and the like), antivirals (for example, valcyclovir), antifungals (for example, miconazole, ketoconazole, clotrimazole, and the like), antiseptics, astringents, vitamins, antioxidants, anti-inflammatories, steroids, anti-irritants and agents to aid in the softening or removal of cerumen.

Formulations of the invention may optionally contain conventional pharmaceutical excipients and preservatives. The term “preservative” refers to an ingredient added to the composition to prevent or retard microbial growth or contamination. Exemplary preservatives include those that are water-soluble and can function as an antimicrobial, such as a benzethonium salt, e.g., benzethonium chloride, parabens, e.g. methylparaben, or organic acids, e.g. sorbic acid. In general, the amount of the preservative ingredient can range from about 0.005-2.0% (w/w, based on the total weight of the formulation).

Buffers, acids, or bases can be added as necessary to adjust the pH of the composition to the preferred range of pH 3-8, most preferably about pH 4.5. Other preservatives and excipients that may be present (generally at less than 2% w/w, or less than 1%, include alkanolamine chlorides, sulfates, phosphates, salts of benzoic acid, acetic acid, salicyclic acid, oxalic acid, phthalic acid, gluconic acid, 1-naphthalenesulfonic acid, 2-naphthalenesulfonic acid, tartaric acid, maleic acid, malonic acid, succinic acid, fumaric acid, propionic acid, ascorbic acid, mandelic acid, malic acid, citric acid, triethanolammonium chloride, triethanolammonium dihydrogen phosphate, triethanolammonium sulfate, sodium benzoate, potassium benzoate, ammonium benzoate, sodium acetate, potassium acetate, ammonium acetate, sodium salicylate, potassium salicylate, ammonium salicylate, sodium oxalate, potassium oxalate, ammonium oxalate, sodium phthalate, potassium phthalate, ammonium phthalate, sodium gluconate, potassium gluconate, ammonium gluconate, ammonium 1-naphthalenesulfonate, potassium 2-naphthalenesulfonate, ammonium 2-naphthalenesulfonate, sodium 2-naphthalenesulfonate, potassium tartarate, sodium maleate, potassium maleate, sodium malonate, sodium succinate, sodium fumarate, sodium propionate, triethanolammonium propionate, sodium ascorbate, triethanolammonium ascorbate, potassium ascorbate, sodium mandelate, sodium malate, sodium citrate, potassium citrate, and triethanolammonium citrate. Chelating agents may also be utilized; e.g., disodium EDTA, edetate trisodium, edetate tetrasodium, or diethyleneamine pentaacetate.

The composition may also contain other active ingredients, such as anti-inflammatories (including, e.g., steroidal compounds (e.g., hydrocortisone, dexamethasone). Suitable compounds and dosages for use in treating pain or inflammation associated with otitis media, such as 0.01-2.0% dexamethasone (e.g., dexamethasone alcohol, dexamethasone acetate or dexamethasone phosphate).

A composition of the invention may optionally also include agents aimed at wound healing including nutrients, growth factors, stem cells, platelets and other cells, small and large molecules, and the like.

In some embodiments, compositions as described herein include one or more compounds anesthetic or analgesic compound. For example, a composition can include one or more pharmacological agents, including, e.g., adrenocorticoid (e.g., corticosteroid, steroid), analgesic, analgesic adjunct, analgesic-anesthetic, anesthetic, antibiotics, antibacterial, anti-infective, antibiotic therapy adjunct, antidote, anti-emetic, anti-fungal, anti-inflammatory, anti-vertigo, anti-viral, biological response modifier, cytotoxic, diagnostic aid, immunizing agent, immunomodulator, proteins, peptides, and other agents that may be useful in treating ear disorders. In addition to an anesthetic or analgesic compound, a composition as described herein can include one or a plurality of pharmacological agents. Those skilled in the art can identify pharmacological agents and combine them as needed to achieve a desired effect. The following simply provides a representative list of possible pharmacological agents.

Exemplary adrenocorticoids include betamethasone, cortisone, dexamethasone, hydrocortisone, methylprednisolone, paramethasone, prednisolone, prednisone, and triamcinolone. Exemplary analgesics include acetaminophen, aspirin, buprenorphine, butalbital, butorphanol, codeine, dezocine, diflunisal, dihydrocodeine, etodolac, fenoprefen, fentanyl, floctafenine, hydrocodone, hydromorphone, ibuprofen, ketoprofen, ketorolac, levorphanol, magnesium salicylate, meclofenamate, mefenamic acid, meperidine, meprobamate, methadone, methotrimeprazine, morphine, nalbuphine, naproxen, opium, oxycodone, oxymorphone, pentazocine, phenobarbital, propoxyphene, salsalate, and sodium salicylate. One exemplary analgesic adjunct is caffeine.

Exemplary antibiotics, anti-bacterials, and anti-infectives include sulfonamides (e.g., sulfanilamide, sulfadiazine, sulfamethoxazole, sulfisoxazole, para-aminobenzoic acid, or sulfacetamide), trimethoprim-sulfamethoxazole, quinolones (e.g., ciprofloxacin, ofloxacin, or nalidixic acid), beta-lactam antibiotics such as penicillins or cephalosporins, aminoglycosides (e.g., kanamycin, tobromycin, gentamycin C, amikacin, neomycin, netilmicin, streptomycin, or vancomycin), tetracyclines, chloramphenicol, and macrolides (e.g., erythromycin, clarithromycin, or azithromycin). Non-limiting examples of suitable penicillins include penicillin G, penicillin V, methicillin, oxacillin, nafcillin, ampicillin, and amoxicillin. Non-limiting examples of suitable cephalosporins include cephalothin, cefdinir, cefazolin, cephalexin, cefadroxal, cefamandole, cefoxitin, cefaclor, cefonicid, cefoletan, cefotaxime, ceftizoxime, ceftriaxone, cefditoren, and cefepime. Exemplary antibiotics useful for treating OM include penicillins such as amoxicillin and amoxicillin-clavulanate (e.g., sold under the tradename AUGMENTIN); sulfa-based combinations such as erythromycin-sulfisoxazole (Pediazole), trimethoprim-sulfamethoxazole (e.g., sold under the tradename BACTRIM or SEPTRA); macrolides/azalides such as azithromycin (e.g., sold under the tradename ZITHROMAX) or clarithromycin (BIAXIN); second-generation cephalosporins such as cefaclor (e.g., sold under the tradename CECLOR), cefprozil (e.g., sold under the tradename CEFZIL), cefuroxime axetil (e.g., sold under the tradename CEFTIN), or loracarbef (e.g., sold under the tradename LORABID; and third generation cephalosporins such as cefdinir (e.g., sold under the tradename OMNICEF), cefixime (e.g., sold under the tradename SUPRAX), cefpodoxime proxetil (e.g., sold under the tradename VANTIN), ceftibuten (e.g., sold under the tradename CEDAX), cefditoren (e.g., sold under the tradename SPECTRACEF), and ceftriaxone (e.g., sold under the tradename ROCEPHIN).

Suitable anti-emetics include buclizine, chlorpromazine, cyclizine, dimenhydrinate, diphenhydramine, diphenidol, domperidone, dronabinol, haloperidol, hydroxyzine, meclizine, metoclopramine, nabilone, ondansetron, perphenazine, prochlorperazine, promethazine, scopolamine, thiethylperazine, triflupromazine, and trimethobenzamine. Exemplary antifungals include amphotericin B, clioquinol, clotrimazole, fluconazole, flucytosine, griseofulvin, ketoconazole, miconazole, and potassium iodide. Exemplary anti-inflammatory agents include aluminum acetate, aspirin, betamethasone, bufexamac, celecoxib, dexamethasone, diclofenac, etodolac, flurbiprofen, hydrocortisone, indomethacin, magnesium salicylate, naproxen, prednisolone, rofecoxib, salsalate, sulindac, and triamcinolone. Exemplary suitable anti-vertigo agents include belladonna, dimenhydrinate, diphenhydramine, diphenidol, meclizine, promethazine, and scopolamine. Exemplary suitable anti-viral agents include acyclovir, amantadine, delavirdine, didanosine, efavirenz, foscarnet, ganciclovir, indinavir, nelfinavir, ribavirin, ritonavir, zalcitabine, and zidovudine. Exemplary biological response modifiers include aldesleukin, interferon alpha-2a, interferon alpha-2b, interferon alpha-n1, interferon alpha-n3, interferon gamma, and levamisole. Exemplary cytotoxic agents include podofilox and podophyllum. Exemplary immunizing agents include influenza virus vaccine, pneumococcal vaccine polyvalent, and immune globulin. An exemplary immunomodulator is interferon gamma. Other pharmacological agents suitable for the invention include betahistine (e.g., for treating the nausea, dizziness, and ringing in the ears that occur in Meniere's disease), prochlorperazine, and hyoscine.

Alternatively or additionally, a composition can include one or more of the following compounds: a solvent or diluent such as saline, a bioadhesive, a permeability or penetraion enhancer, a hygroscopic agent, an earwax softener, preservative (e.g., an antioxidant), or other additives. Such compounds can be present in the composition in amounts ranging from 0.01% to 99% (e.g., 0.01 to 1, 0.01 to 10, 0.01 to 40, 0.01 to 60, 0.01 to 80, 0.5 to 10, 0.5 to 40, 0.5 to 60, 0.5 to 80, 1 to 10, 1 to 40, 1 to 60, 1 to 80, 5 to 10, 5 to 40, 5 to 60, 5 to 80, 10 to 20, 10 to 40, 10 to 60, 10 to 80, 20 to 30, 30 to 40, 40 to 50, 50 to 60, 60 to 70, or 70 to 80%). For example, a composition can include one or more viscogenic agents (e.g., PLURONIC F-127 and CARBOPOL), an anesthetic or analgesic compound and one or more permeability or penetration enhancers (e.g., vitamin E). In other embodiments, a composition can include one or more viscogenic agents, and anesthetic or analgesic compound, and one or more earwax softeners. Compositions also can include one or more viscogenic agents, an anesthetic or analgesic compound, one or more hygroscopic agents, and one or more preservatives. It is noted that certain agents can fulfill different roles within the formulation. For example, CARBOPOL can function as a viscogenic agent or as a bioadhesive, depending on its concentration. Vitamin E can function as a permeability or penetration enhancer, a preservative, and an antioxidant.

A bioadhesive can facilitate the adhesion of the composition to the tympanic membrane. Suitable bioadhesives include hydrocolloids such as: acacia; agar agar; alginates (e.g., alginic acid and sodium alginate); CABOPOL; carboxymethylcellulose sodium; carboxymethylcellulose calcium; dextran; gelatin; guar gum; heparin; hyaluronic acid; hydroxyethylcellulose; karaya gum; methylcellulose; pectin; polyacrylic acid; polyethylene glycol; poly-N-vinyl-2-pyrrolidone; and tragacanth.

Permeability or penetration enhancers increase the permeability of the tympanic membrane to make it more permeable to an active agent. Exemplary permeability or penetration enhancers include: alcohols (e.g., ethanol and isopropanol); polyols (e.g., n-alkanols, limonene, terpenes, dioxolane, propylene glycol, ethylene glycol, and glycerol); sulfoxides (e.g., dimethylsulfoxide, dimethylformamide, methyl dodecyl sulfoxide, and dimethylacetamide); esters (e.g., isopropyl myristate/palmitate, ethyl acetate, butyl acetate, methyl proprionate, and capric/caprylic triglycerides); ketones; amides (e.g., acetamides); oleates (e.g., triolein); surfactants (e.g., sodium lauryl sulfate); alkanoic acids (e.g., caprylic acid); lactams (e.g., azone); alkanols (e.g., oleyl alcohol); dialkylamino acetates; polyunsaturated fatty acids (e.g., linoleic, alpha-linolenic, and arachidonic); oleic acid; cod-liver-oil; menthol derivatives (e.g., 1-menthol); Squalene; glycerol monoethers derived from linear saturated fatty alcohols; flavones (e.g., chamomile apigenin, luteolin, and apigenin 7-O-beta-glucoside); vitamin E (.alpha.-tocopherol) and esters and analogs thereof; and Senkyu (Ligustici Chuanxiong Rhizome) ether extract.

Hygroscopic agents such as fructose, phthalic acid, and sorbitol, facilitate the transfer of fluid from the middle ear across the tympanic membrane into the gel matrix. Hygroscopic agents can help alleviate pain associated with fluid accumulation and pressurization of the middle ear.

Earwax softeners (e.g., docusate, olive oil, sodium bicarbonate, urea, or hydrogen peroxide) facilitate contact between the tympanic membrane and the composition. An antioxidant such as ascorbic acid and benzoic acid or other preservatives can be used to extend the shelf life of the formulation during storage.

In certain embodiments, the composition is substantially free of alcohol and/or zinc ions. In certain embodiments, the composition is substantially free of membrane penetrator compounds. In certain embodiments, the composition is substantially free of antibiotics.

### Unit Dosage Forms

In another aspect, the invention provides a pharmaceutical composition comprising a topical formulation comprising:

(a) an effective pain-reducing amount of an anesthetic or analgesic; and

(b) a pharmaceutically acceptable carrier;

wherein the topical formulation is substantially non-flowable after application to an ear canal of a subject. The pharmaceutical composition can be provided in unit dosage form.

A formulation of the invention can be provided as a unit dosage form in a hermetically sealed container such as an ampoule or sachette indicating the quantity of active agent. Where the composition is to be administered by instillation into the ear canal, it can be dispensed with a dropper, syringe, infusion bottle, or other suitable applicator. Alternatively, the hermetically sealed container can be a prefilled syringe or dropper, in which a seal is broken to permit use of the syringe for application of the composition to the ear canal.

In certain embodiments, the composition can be provided as a dry powder concentrate or liquid or semiliquid concentrate, to which water or another pharmaceutically acceptable solvent is added to solubilize or suspend the components of the formulation. In such embodiments, the unit dosage form can include instructions for dilution or reconstitution of the formulation prior to use.

In certain embodiments, the volume of the unit dosage form is less than 1.0 cc, or less than 0.9 cc, or less than 0.8 cc, or less than 0.7 cc, or less than 0.6 cc, or less than 0.5 cc, or less than 0.4 cc, or less than 0.3 cc.

### Spray Formulations

A formulation of the invention may be applied to the ear canal with a dropper, a solid applicator, an atomizer, or a sprayer. Conventional applicators, such as sprayers, can be selected to provide suitable amounts and placement of the formulation within the ear canal. The choice of a suitable applicator and method of application is routine for the ordinarily-skilled artisan. The formulations can be stored in a single-dose or multi-dose container prior to application. A formulation can be applied so as to be substantially non-flowable, e.g., non-leaking, by applying a small volume of material that is well dispersed (in a thin layer) over the ear canal and/or tympanic membrane.

### Methods for Treating Ear Pain

In another aspect, the invention provides methods for treating ear pain, e.g., pain due to otitis, e.g., otitis media, or other conditions of the external, middle or inner ear, or other bodily organs or structures accessible from the ear canal.

In certain embodiments, the method comprises administering a composition of the invention to an ear canal of a subject in need of such treatment. In certain embodiments, after 5 minutes after application, the composition does not flow from the ear canal for at least about 10 minutes (or at least about 30 minutes, one hour, and the like). In certain embodiments, the ear pain is pain due to otitis.

In certain embodiments, the method comprises administering a composition of the invention to an ear canal of a subject in need of such treatment, under conditions such that leakage of the composition from the ear canal is minimized without plugging the ear canal.

As used herein, the term “subject” or “patient” refers to a warm-blooded animal, such as a mammal, including primates (including humans), cats, dogs, sheep, goats, cattle, pigs, rodents (including without limitation rats, mice, hamsters, guinea pigs, gerbils, and rabbits) and the like.

In general, a composition of the invention is delivered into the ear canal, e.g., into the middle ear, by any of a variety of methods, some of which are known in the art. For example, a composition can be administered via any medically acceptable means for application of a pharmaceutical composition to the ear canal, e.g., by insertion of a needleless syringe or dropper into the auditory canal. It will be appreciated that care should be used to avoid piercing or puncturing the tympanic membrane.

Administration is repeated as required to achieve the therapeutically effective dosage level for the anesthetic or analgesic compound and/or other medicament(s) given. In certain embodiments, the amount of analgesic or anesthetic to be administered to the ear canal is between about 1 mg and about 100 mg per dose. An exemplary regimen of dosing with the formulation described in Example 1 herein is 0.3 mL twice a day for a child under age 12, and 0.6 mL twice a day for an adult or a child of age 12 or older.

In certain embodiments, the volume of the composition that is administered or applied to the ear canal is less than the volume of the ear canal. For example, an adult ear canal may have a volume from about 0.6 cubic centimeters (cc) to about 1.5 cc, and a child's ear canal may have a volume from about 0.4 cubic centimeters (cc) to about 1.0 cc. Thus, in certain embodiments, the volume of the composition that is administered or applied to the ear canal is between about 0.6 cc and about 1.5 cc, or is between about 0.4 cc and about 1.0 cc, or is between about 0.1 cc and about 0.5 cc. In certain embodiments, the volume of the composition that is administered or applied to the ear canal is less than 1.0 cc, or less than 0.9 cc, or less than 0.8 cc, or less than 0.7 cc, or less than 0.6 cc, or less than 0.5 cc, or less than 0.4 cc, or less than 0.3 cc, or less than 0.2 cc, or or less than 0.1 cc.

In certain embodiments, the subject experiences the onset of pain relief rapidly after administration, e.g., within 10 minutes of application, or within 5 minutes of application, or most preferably within 1 minute of application of the formulation to the ear canal.

In certain embodiments, the subject experiences pain relief (i.e., the analgesic and/or anesthetic is effective) for at least about 15 minutes after application, or at least about 30 minutes, one hour, two hours, four hours, or six hours after application of the formulation to the ear canal.

In certain embodiments, the product is refrigerated prior to administration.

Prophylactic treatment against recurrence of a middle ear infection may be provided in the same manner, utilizing a composition of the invention containing a prophylactically effective antibiotic or other medicament.

Those of ordinary skill in the art will be familiar with, and readily able to select, dosing regimens suitable for following to treat a particular ear pain. The dosing regimen selected will be in accord with established clinical protocols for delivery and use of the particular carrier and medicaments provided according to the invention.

### EXAMPLES

The following examples are intended to illustrate, but not to limit, the invention.

### Example 1

### 0.3 mL Dose Suspension (Volatile Base)

An exemplary suspension having a volatile carrier can be prepared as follows:

Combine 10 g clove oil and 65 g Cyclomethicone 5 and mix until homogeneous. Add 16.2 g antipyrine and 4.2 g benzocaine and process on a Silverson rotor/stator L4RT-A lab mixer with a ⅝″ micro head at 2500 rpm for 5 minutes with cooling. Q.S. to 100 mL with Cyclomethicone 5 and mix until homogeneous.

The suspension is shaken before instillation. Upon administration to the ear canal, the volatile material evaporates, leaving a viscous residue that remains in the inner ear.

### Example 2

### 0.3 mL Dose Suspension (Volatile Base) with Acetaminophen

An exemplary suspension having a volatile carrier can be prepared as follows:

Combine 10 g mineral oil and 60 g Cyclomethicone 5 and mix until homogeneous. Add 16.2 g antipyrine, 4.2 g benzocaine, and 5 g acetaminophen, and process on a Silverson rotor/stator L4RT-A lab mixer with a ⅝″ micro head at 2500 rpm for 5 minutes with cooling. Q.S. to 100 mL with Cyclomethicone 5 and mix until homogeneous.

The suspension is shaken before instillation. Upon administration to the ear canal, the volatile material evaporates, leaving a viscous residue that remains in the inner ear.

Alternatively, APIs can be jet-milled prior to use to reduce particle size.

### Volatile Suspension Applicable Ingredients

| | % range | Primary | Secondary |
|---|---|---|---|
| Ingredient | (w/v) | Function | Function |
| Antipyrine | 1-40 | Analgesic | |
| Benzocaine | 1-10 | Anesthetic | |
| Acetaminophen | | Analgesic | |
| Amoxicillin | | Antibiotic | |
| Cefdinir | | Antibiotic | |
| Azithromycin | | Antibiotic | |
| Clarithromycin | | Antibiotic | |
| Cefuroxime | | Antibiotic | |
| Ceftriaxone | | Antibiotic | |
| Benzyl Alcohol | 0-5 | Cerumen | |
| | | Removal | |
| Cyclomethicone 5, NF | 85-99 | Volatile Carrier | |
| Dimethicone | 0-10 | Carrier | Skin Protectant |
| Isopropyl Myristate | 0-10 | Carrier | Emollient |
| Isopropyl Palmitate | 0-10 | Carrier | Emollient |
| Octyl Palmitate | 0-10 | Carrier | Emollient |
| Capric/Caprylic | 0-10 | Carrier | Emollient |
| Triglycerides | | | |
| Mineral Oil | 0-10 | Carrier | Emollient |
| Clove Oil | 0-10 | Carrier | Anesthetic |
| Soybean Oil | 0-10 | Carrier | Emollient |

### Example 3

### 1 mL Dose Volatile Emulsion

An exemplary emulsion having a volatile carrier can be prepared as follows:

Combine 10 g PEG1000, 10 g propylene glycol, 5 g purified water and 0.1 g NaCl. Mix and heat to 50° C. until homogeneous. Add 5.4 g antipyrine and 1.4 g benzocaine and continue mixing until dissolved (Phase A). Separately, combine 0.5 g Dimethicone 350, 1.5 g Cetyl PEG/PPG-10/1 Dimethicone, and 60 g Cyclomethicone 5 (Phase B). Mix and heat to 50° C. until homogeneous. With continued mixing, add Phase A to Phase B. Insert horn tip of ultrasonic processor (450 watt) into mixture and process at full power for 2-3 minutes. Remove horn tip and cool to 25 C. Q.S. to 100 mL with Cyclomethicone 5 and mix until homogeneous.

### Volatile Emulsion Applicable Ingredients

| | % range | Primary | Secondary |
|---|---|---|---|
| Ingredient | (w/v) | Function | Function |
| Antipyrine | 1-40 | Analgesic | |
| Benzocaine | 1-10 | Anesthetic | |
| Acetaminophen | | Analgesic | |
| Amoxicillin | | Antibiotic | |
| Cefdinir | | Antibiotic | |
| Azithromycin | | Antibiotic | |
| Clarithromycin | | Antibiotic | |
| Cefuroxime | | Antibiotic | |
| Ceftriaxone | | Antibiotic | |
| Benzyl Alcohol | 0-5 | Preservative | Cerumen Removal |
| Cyclomethicone 5, NF | 85-99 | Volatile Carrier | |
| Glycerin | 0-15 | Solvent | |
| Polyethylene Glycol | 0-75 | Solvent | |
| Propylene Glycol | 0-95 | Solvent | |
| Water | 0-50 | Solvent | |
| Dimethicone Copolyol | 0-10 | Emulsifier | |
| NaCl | 0-1 | Stabilizer | |
| Dimethicone | 0-10 | Carrier | Skin Protectant |
| Isopropyl Myristate | 0-10 | Carrier | Emollient |
| Isopropyl Palmitate | 0-10 | Carrier | Emollient |
| Octyl Palmitate | 0-10 | Carrier | Emollient |
| Capric/Caprylic | 0-10 | Carrier | Emollient |
| Triglycerides | | | |
| Mineral Oil | 0-10 | Carrier | Emollient |
| Clove Oil | 0-10 | Carrier | Anesthetic |
| Soybean Oil | 0-10 | Carrier | Emollient |

### Example 4

### 0.5 mL Dose Shear Responsive Thinning Gel

An exemplary shear responsive thinning gel can be prepared as follows:

Dissolve 2.8 g benzocaine in 25 g propylene glycol (Phase A), mix until dissolved. Add 10.8 g antipyrine, 0.2 g polysorbate 80, and 0.9 g benzyl alcohol to 75 g purified water and mix until dissolved (Phase B). With vigorous mixing, add Phase A to Phase B. Mix until well dispersed. With continued mixing slowly add 0.15% Carbomer 934P and mix until dissolved and homogeneous (NLT 60 min). Add 5N NaOH to adjust pH to 6.8-7.2 and thicken the preparation. Q.S. to 100 mL with purified water and mix until homogeneous.

The gel can be shaken to reduce viscosity to ease instillation and will return to a higher viscosity upon standing after application.

### Example 5

### 0.5 mL Dose Shear Responsive Thinning Gel with Antibiotic

An exemplary shear responsive thinning gel including an antibiotic can be prepared as follows:

Dissolve 2.8 g benzocaine in 25 g propylene glycol (Phase A), mix until dissolved. Add 10.8 g antipyrine, 0.25 g amoxicillin, 0.2 g Polyoxyl 40 Stearate, and 0.9 g benzyl alcohol to 75 g purified water and mix until dissolved (Phase B). With vigorous mixing, add Phase A to Phase B. Mix until well dispersed. With continued mixing slowly add 0.05% Carbomer 934P and 0.1% hydroxyethylcellulose and mix until dissolved and homogeneous (NLT 60 min). Add 5N NaOH to adjust pH to 6.8-7.2 and thicken the preparation. Q.S. to 100 mL with purified water and mix until homogeneous.

The gel can be shaken to reduce viscosity to ease instillation and will return to a higher viscosity upon standing after application.

### Example 6

### 0.5 mL Dose Thermogel

An exemplary thermogel can be prepared as follows:

Dissolve 2.8 g benzocaine in 25 g glycerin (Phase A), mix until dissolved. Add 10.8 g antipyrine and 0.9 g benzyl alcohol to 50 g purified water and mix until dissolved (Phase B). With vigorous mixing, add Phase A to Phase B. Mix until well dispersed. Cool Phase NB to 5 C. With continued mixing add 10 g poloxamer 407 and mix until completely dissolved. Q.S. to 100 mL with purified water and mix until homogeneous. Allow to warm to 25° C. to form gel.

The gel can be cooled to reduce viscosity and will gel further upon instillation and exposure to body temperature (37° C.).

Alternatively, the amount of Poloxamer gelling agent can be reduced and a small quantity of water-soluble thicker (Carbomer or the like) can be included to modulate viscosity.

### Shear Responsive Thinning Gel and Thermogel Applicable Ingredients

| | % range | Primary | Secondary |
|---|---|---|---|
| Ingredient | (w/v) | Function | Function |
| Antipyrine | 1-40 | Analgesic | |
| Benzocaine | 1-10 | Anesthetic | |
| Acetaminophen | | Analgesic | |
| Amoxicillin | | Antibiotic | |
| Cefdinir | | Antibiotic | |
| Azithromycin | | Antibiotic | |
| Clarithromycin | | Antibiotic | |
| Cefuroxime | | Antibiotic | |
| Ceftriaxone | | Antibiotic | |
| Poloxamer 407 | 0-40 | Dispersant | Gelling agent |
| Polysorbates | 0-5 | Dispersant | |
| Polyoxyl stearates | 0-5 | Dispersant | |
| Povidone | 0-10 | Dispersant | |
| Carbomer | 0-1 | Thickener | |
| Bentonite | 0-30 | Thickener | |
| Hydroxyethylcellulose | 0-5 | Thickener | |
| Hydroxypropylcellulose | 0-5 | Thickener | |
| Sodium | 0-5 | Thickener | |
| Carboxymethylcellulose | | | |
| Glycerin | 0-75 | Solvent | |
| Polyethylene Glycol | 0-75 | Solvent | |
| Propylene Glycol | 0-95 | Solvent | |
| Parabens | 0-0.5 | Antimicrobial | |
| Sorbic acid | 0-2.5 | Antimicrobial | |
| Benzyl Alcohol | 0-5 | Antimicrobial | Antipuritic |
| Sodium hydroxide | 0-0.5 | pH modifier | |
| Triethanolamine | 0-5 | pH modifier | Cerumen |
| | | | removal |

All patents, patent applications, and published references cited herein are hereby incorporated by reference in their entirety.

While this invention has been particularly illustrated and described with reference to particular examples, it will be understood by those skilled in the art that various changes in form and details may be made therein without departing from the scope and spirit of the invention encompassed by the appended claims.

What is claimed is:

1. A topical formulation comprising:
(a) an effective pain-reducing amount of an anesthetic or analgesic; and
(b) a pharmaceutically acceptable carrier;
wherein the topical formulation is substantially non-flowable after application to an ear canal of a subject.

2. The formulation of claim 1, wherein the anesthetic or analgesic is selected from the group consisting of antipyrine, benzocaine, lidocaine, butamben, dibucaine, oxybuprocaine, pramoxine, proparacaine, proxymetacaine, and tetracaine.

3. The formulation of claim 1, wherein the anesthetic is an essential oil having anesthetic properties, or an active component of such an essential oil.

4. The formulation of claim 3, wherein the essential oil is clove oil or fennel oil.

5. The formulation of claim 3, wherein the active component is eugenol, linalool, or fenchone, or a mixture thereof.

6. The formulation of claim 1, further comprising a second therapeutic agent.

7. The formulation of claim 6, said second therapeutic agent being selected from the group consisting of: antibiotics, antivirals, antifungals, antiseptics, astringents, anti-irritants, anti-inflammatories, and agents to aid in the softening or removal of cerumen.

8. The formulation of claim 1, wherein the active ingredient or ingredients are present as a particle, capsule or solution, or a combination thereof.

9. The formulation of claim 1, wherein the formulation has a viscosity of at least about 10,000 cps.

10. The formulation of claim 1, wherein the anesthetic is dispersed or dissolved in a volatile carrier.

11. The formulation of claim 10, wherein the volatile carrier comprises a material selected from the group consisting of volatile silicones, alcohols, biodegradable polymers, or volatile oils.

12. The formulation of claim 1, wherein the volatile carrier comprises cyclomethicone 5.

13. The formulation of claim 1, wherein the formulation is shear thinning such that the formulation can be thinned for application by shaking and thickens when applied to the ear canal.

14. The formulation of claim 1, wherein the formulation thickens upon application to the ear canal.

15. The formulation of claim 1, wherein the formulation has a low viscosity when chilled and is substantially non-flowable at body temperature.

16. The formulation of claim 15, wherein the formulation comprises a viscosity adjusting agent, wherein the viscosity adjusting agent comprises a polymer which exhibits a thermal gelation response.

17. The formulation of claim 16, wherein the viscosity adjusting agent comprises a polymer selected from the group consisting of chitosan, cellulosic derivatives, gelatin, N-isopropylacrylamide polymers, poly(ethylene oxide)-b-poly(propylene oxide)-b-poly(ethylene oxide) polymers and poly(ethylene glycol)-biodegradable polyester copolymers.

18. The formulation of claim 1, wherein the formulation further comprises a drying agent.

19. A topical formulation, the formulation comprising an anesthetic, wherein the formulation provides anesthesia for at least about 4 hours after application to an ear canal of a subject.

20. A topical formulation, the formulation comprising an anesthetic, wherein the formulation has an onset of action of less than about 10 minutes after application to an ear canal of a subject.

21. The formulation of claim 1, wherein the formulation is essentially free of zinc.

22. The formulation of claim 1, wherein the formulation is essentially free of additional penetration enhancers.

23. The formulation of claim 1, wherein the formulation is essentially free of additional antibiotics.

24. The formulation of claim 1, wherein the formulation is essentially free of additional preservatives.

25. A method of treating ear pain, the method comprising administering a composition of claim 1 to an ear canal of a subject in need of such treatment.

26. The method of claim 25, wherein, after 5 minutes after application, the composition does not flow from the ear canal for at least about 10 minutes.

27. The method of claim 25, wherein the ear pain is pain due to otitis.

28. A method of treating ear pain, the method comprising administering a composition of claim 1 to an ear canal of a subject in need of such treatment, under conditions such that leakage of the composition from the ear canal is minimized without plugging the ear canal.

29. A topical formulation consisting essentially of:
(a) an effective pain-reducing amount of an anesthetic or analgesic; and
(b) a pharmaceutically acceptable carrier;
wherein the topical formulation is substantially non-flowable after application to an ear canal of a subject.

30. A topical formulation comprising:
(a) an effective pain-reducing amount of an anesthetic or analgesic; and
(b) a pharmaceutically acceptable carrier;
wherein the topical formulation does not leak from the ear canal of a subject after application of the formulation to the ear canal of the subject.
